(19) United States
(12) Patent Application Publication
(10) Pub. No.: US 2011/0196220 A1
(43) Pub. Date: Aug. 11, 2011

(54) Electrode Assembly for Medical Purposes

(75) Inventor: Hanne Storm, Oslo (NO)

(73) Assignee: MED STORM INNOVATION AS, Oslo (NO)

(21) Appl. No.: 13/122,734

(22) PCT Filed: Oct. 22, 2009
(86) PCT No.: PCT/NO09/00365

§ 371 Date: Apr. 6, 2011

(30) Foreign Application Priority Data
Oct. 22, 2008 (NO) 20084440

(51) Int. Cl. A61B 5/04

(52) U.S. Cl. 600/393

(57) ABSTRACT

An electrode assembly for medical purposes comprises a to first electrode 11electrically connected to a first connecting line 15, a second electrode 12electrically connected to a second connecting line 16, and a third electrode 13electrically connected to a third connecting line 17. The first, second and third electrodes are GI arranged in a single patch 14. The shape or area of the second electrode is different from the shape or area of the first and third electrodes. The first, second and third connection lines may be coded by visible or tactile coding means.

### FIELD OF THE INVENTION

The invention relates to an electrode assembly for medical purposes, particularly to be placed on the skin of a patient.

### BACKGROUND OF THE INVENTION

Medical electrodes are needed in many applications, e.g. for measurement of electrical activity in the brain (EEG), the heart (ECG), or the muscles (EMG). Medical electrodes may be used for measuring, e.g., electric current, potential/voltage, skin conductance, and skin resistance. Certain medical electrodes may also be used for supplying electrical energy into the patient, e.g. in the case of defibrillators, electroconvulsive therapy and other therapy methods.

Medical electrodes are also used in the fields of electrophysiology and neuroscience. A particular class of equipment and methods utilize measurement of skin conductance for assessing the state of the autonomous nervous system of a patient, such as a sedated patient, e.g. for detecting pain or awakening during anesthesia.

WO-03/094726 is an example of an apparatus and a method for monitoring the autonomous nervous system of a sedated patient, wherein the skin conductance, measured by use of electrodes placed on the skin of the patient, is measured. Signals indicating pain/discomfort in the patient and awakening in the patient are derived from the skin conductance measurements.

### SUMMARY OF THE INVENTION

The invention is set forth in the appended claims.

### BRIEF DESCRIPTION OF THE DRAWINGS

Embodiments, aspects and principles of the invention will now be described in more detail with reference to the attached drawings, in which

FIG. 1 is a schematic block diagram illustrating an electrode assembly in a first embodiment, and

FIG. 2 is a schematic block diagram illustrating an electrode assembly in a second embodiment.

### DETAILED DESCRIPTION

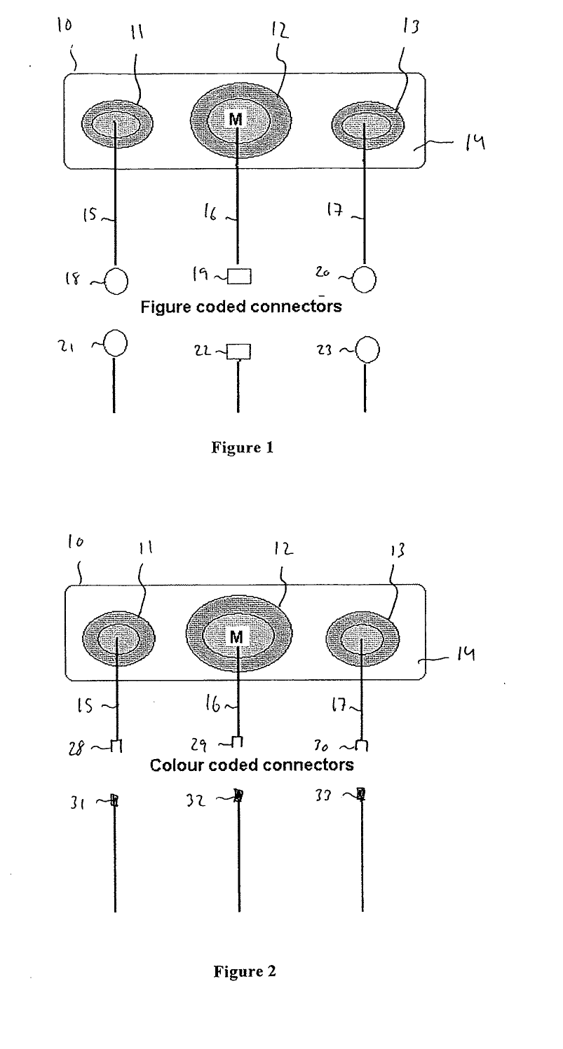

FIG. 1 is a schematic block diagram illustrating an electrode assembly in a first embodiment.

The electrode assembly 10 comprises a first electrode 11, a second electrode (M) 12, and a third electrode 13, all arranged in a patch 14.

In an aspect, the first electrode 11 is a counter current electrode, the second electrode 12 is a measuring electrode, and the third electrode 13 is a reference voltage electrode.

The first electrode 11 is electrically connected to a first connecting line 15, which is further connected to a first connection device 18. Similarly, the second electrode 12 is electrically connected to a second connecting line 16, further connected to a second connection device 19. Similarly, the third electrode 13 is electrically connected to a third connecting line 17, further connected to a third connection device 20.

In the illustrated example, the area of the second electrode 12 is different from the area of the first 11 and second 13 electrodes.

In an aspect, the shapes of the first and third electrodes are the same. In a particular aspect, the shape of the second electrode may be the same as the shapes of the first electrode and the third electrode. In another particular aspect, the shape of the second electrode may be different from the shapes of the first electrode and the third electrode.

In the illustrated example, the first 11, second 12 and third 13 electrodes have oval shapes. Alternatively, they could have circular shapes, or other shapes such as square, rectangular, triangular, etc.

In an aspect, as illustrated, the area of the first 11 and the third 13 electrodes may be the same, and the area of the second electrode 12 may be larger than the area of the first 11 and second 13 electrodes.

The patch 14 may have an elongated shape, e.g. a substantially rectangular shape as illustrated.

In an aspect, the second electrode 12 is a measurement electrode 12 and is arranged in between the first (counter current) electrode 11 and the third (reference voltage) electrode 13.

In an aspect, the first electrode 11 and the third electrode 13 are arranged substantially symmetrically about a line perpendicular to the length of the longitudinal patch 14.

In an aspect, the first electrode 11, the second electrode 12 and the third electrode 13 are arranged substantially in-line.

In another aspect, the first electrode 11, the second electrode 12 and the third voltage electrode 13 are not arranged in-line.

The first 15, the second 16 and the third 17 connection lines may be coded by visible or tactile coding means.

In an aspect, as schematically illustrated in FIG. 1, the coding means are both visible and tactile. In this case the first 15, second 16 and third 17 connection lines are figure coded. This is achieved by providing the first 15 and third 17 connection lines with connectors 18, 20 of a first type and by providing the second connection line 16 with a connector 19 which is incompatible with the connectors 18, 20 of the first type.

As an example, the connectors 18 and 20 may be a female connector, i.e. a socket, with a circular cross-section, while the connector 19 is a female connector, i.e. a socket, with a rectangular or square cross-section.

Correspondingly, wires for connection to measurement equipment (not shown) are provided with male connector (plug or pin type) with appropriate circular cross-section (connectors 21, 23), and a male connector (plug or pin type) with appropriate rectangular or square cross-section (connector 22).

FIG. 2 is a schematic block diagram illustrating an electrode assembly in a second embodiment.

Please refer to the description of FIG. 1 for a detailed description of those elements of FIG. 2 which are identical to (and have the same reference numerals as) those illustrated in FIG. 1.

In the embodiment of FIG. 2, the first 15, second 16 and third 17 connection lines are coded by visible coding means. This is achieved by color coding the connection lines. By example, this may be achieved by using different colors of the wires 15, 16, and 17, and/or the connectors 28, 29, 30, and/or the connectors 31, 32, 33.

By example, a blue insulation may be used for the second connection line 32, while red insulation is used for the first 31 and third 33 connection lines.

In such an embodiment, the connectors 28, 29, 30 may be of the same shape and type, e.g. female (socket) connectors, corresponding to male (plug/pin) connectors 31, 32, 33, respectively.

The electrode assembly disclosed in any of the above embodiments may be used in an arrangement for measuring skin conductance, e.g. of a patient.

In such use, the electrode assembly 10 may be placed on an area of the skin on a body part of the patient, for the purpose of measuring the skin's conductance. The body part may, e.g., be a hand or a foot, and the area of the skin on the body part may, e.g., be the palmar side of the hand or the plantar side of the foot.

The first electrode 11 is a counter current electrode, the second electrode 12 is a measuring electrode 12, and the third electrode 13 is a reference voltage electrode, all arranged in the patch 14, which is placed on the skin area.

The electrode assembly 10 and its connected external equipment may ensure a constant application of voltage over the stratum corneum (the surface layer of the skin) under the measuring electrode.

The resulting current in a closed circuit between the measuring electrode 12 and the counter current electrode 11 may be measured by means of a measurement converter (not shown), connected between the counter current electrode 11 and the measuring electrode 12. The measurement converter may comprise a current to voltage converter, such as a transresistance amplifier or simply a resistance, which converts the current from the measuring electrode 12 to a voltage.

The arrangement of a stiff patch 14 ensures that the second electrode (or measuring electrode) 12 does not suffer from undesired movement, which might lead to artefacts in the resulting measurements.

The arrangement of the measuring electrode 12 in the middle also contribute to reduced artefacts.

Since the skin conductance changes are monitored below the measuring electrode 12, the area below the measuring electrode 12 may be critical.

Further possible values and specifications of the electrode assembly and its elements are given below (for illustration, not for limitation):

### Sensor:

Single use self-adhesive Ag/AgCl

Solid gel

The measuring sensor area (M) may be between 260 and 480 mm²

Operational principle; 3 measuring points, minimum 1 cm between the measuring points.

### Dimension:

Electrode size and skin conduct size with adhesive area may have a minimum area of 260 mm²,

Connecting wire between 1 and 1000 mm.

The electrodes do not necessarily need to be on a line PS Materials:

Biocompatible

Color coded or figure coded for ease of identification

Outer carrier, e.g. polyester

Lead-wire insulation, e.g. polyvinyl chloride

Lead-wire connector, e.g. polypropylene

The electrode assembly disclosed herein may also be used in an arrangement for monitoring the autonomous nervous system of a patient.

In this case a similar arrangement is used as explained above. In addition, the measuring equipment is connected to a processing device configured for monitoring the autonomous nervous system of the patient, based on measurements of the skin conductance. An example of suitable processing and monitoring means/methods has been disclosed in WO-03/094726 (expressly incorporated by reference).

The inventive concept is not limited to the exemplary embodiments described above. Rather, the scope of the invention is set forth in the following patent claims.

1. Electrode assembly for medical purposes,
comprising a first electrode electrically connected to a first connecting line,
a second electrode electrically connected to a second connecting line, and
a third electrode electrically connected to a third connecting line,
the first, second and third electrodes being arranged in a single patch,
wherein the shape or area of the second electrode is different from the shape or area of the first and third electrodes, and
wherein the first, second and third connection lines are coded by tactile coding means.

2. Electrode assembly according to claim 1,
wherein the shapes of the first and third electrodes are the same.

3. Electrode assembly according to claim 1,
wherein the area of the first and third electrodes are the same.

4. Electrode assembly according to claim 3,
wherein the area of the second electrode is larger than the area of the first and third electrodes.

5. Electrode assembly according to claim 1,
wherein said first, second and third connection lines are figure coded,
the first and third connection lines being provided with a connector of a first type and the second connection line being provided with a connector which is incompatible with the connector of the first type.

6. Electrode assembly according to claim 1, wherein said first, second and third connection lines are color coded.

7. Electrode assembly according to claim 1, wherein
said first electrode is a counter current electrode,
said second electrode is a measuring electrode, and
said third electrode is a reference voltage electrode.

8. Use of an electrode assembly according to claim 1
in an arrangement for measuring skin conductance.

9. Use of an electrode assembly according to claim 1 in an arrangement for monitoring the autonomous nervous system of a patient.

10. Electrode assembly according to claim 7,
wherein the measuring electrode is arranged in the middle, between the counter current electrode and the reference voltage electrode.

11. Electrode assembly according to claim 10,
wherein the area of the measuring electrode is between 260 mm² and 480 mm².

12. Electrode assembly according to claim 1,
wherein the shapes of the first and third electrodes are the same,
wherein the area of the first and third electrodes are the same,
wherein the area of the second electrode is larger than the area of the first and third electrodes,
wherein said first, second and third connection lines are figure coded,
the first and third connection lines being provided with a connector of a first type and the second connection line being provided with a connector which is incompatible with the connector of the first type.

13. Electrode assembly according to claim 12, wherein
said first electrode is a counter current electrode,
said second electrode is a measuring electrode,
said third electrode is a reference voltage electrode,
the measuring electrode is arranged between the counter current electrode and the reference voltage electrode, and
the area of the measuring electrode is between 260 mm² and 480 mm².
